(19) United States
(12) Patent Application Publication
(10) Pub. No.: US 2020/0138714 A1
(43) Pub. Date: May 7, 2020

(54) Quickly Disintegrating Foam Wafer with High Mass Per Unit Area

(71) Applicant: LTS Lohmann Therapie-Systeme AG, Andemach (DE)

(72) Inventors: Markus MÜLLER, Troisdorf (DE); Marius BAUER, Andernach (DE); Michael LINN, Waldböckelheim (DE)

(21) Appl. No.: 16/619,359

(22) PCT Filed: Jun. 7, 2018
(86) PCT No.: PCT/EP2018/065008

§ 371 Date: Dec. 4, 2019

(30) Foreign Application Priority Data
Jun. 7, 2017 (DE) 102017112527.2

(51) Int. Cl. A61K 9/12; A61K 31/135; A61K 31/439; A61K 47/02

(52) U.S. Cl. CPC A61K 9/122; A61K 47/02; A61K 31/439; A61K 31/135

(57) ABSTRACT

The invention relates to flat dosage forms that disintegrate or dissolve in an aqueous environment and release at least one active ingredient in a body orifice or body cavity. The inventive dosages are formed from a polymer matrix in the form of a solidified foam having cavities and contain at least one pharmaceutical active ingredient. The inventive dosage forms further have a high mass per unit area in the region of 50 to 350 g/m². Despite their high mass per unit area and the resulting high active ingredient loading during administration, these dosage forms exhibit a substantially improved mouthfeel in comparison to conventional film dosage forms. The invention also relates to methods for producing a dosage form of this type.

The invention relates to a planar dosage form that disintegrates or dissolves in an aqueous environment for releasing active ingredients which can be administered especially orally by means of the dosage form and which have a matrix based on water-soluble polymers as base substances. Especially the invention relates to dosage forms of the described kind which are shaped in the form of wafers. The invention also relates to methods for producing such dosage forms.

### PRIOR ART

In order to administer active ingredients via the oral mucosa, buccal or sublingual tablets are usually used, which release the active ingredient in the oral cavity. The resorption of the active ingredient via the oral mucosa offers a number of advantages compared to other peroral dosage forms, for example the fact that medicaments can be administered orally also to patients who have difficulty swallowing, the fact that the effect takes hold quickly due to a bypassing of the gastrointestinal passage, and the fact that the active ingredient utilisation is high.

Planar, wafer-like dosage forms, also referred to as wafers, are known as alternative dosage forms to the known buccal and sublingual tablets.

For example, U.S. Pat. No. 5,529,782 describes a rapidly dissolving film product formed from soluble polymer material or complex polysaccharides, which is used predominantly for the administration of contraceptives. The film product should have a thickness of from 3 to 4.5 mm, and it should be possible to set its solubility such that the film product will have dissolved within 5 to 60 seconds after administration. The film product may also be present in the form of a laminate, which has cavities foamed with gas.

A carrier material for administering medicinal products which dissolves rapidly upon contact with saliva is known from EP 0450 141 B1. This carrier material is a porous, dehydrated, skeleton-like carrier material, especially based on proteins and polysaccharides. The cavities produced by dehydration are used in order to introduce liquid active ingredients.

WO 98/26764 describes an active-ingredient-containing and film-like dosage form that disintegrates rapidly upon contact with liquid, wherein a fat-soluble phase is distributed in the form of liquid droplets in an outer water-soluble phase.

WO 00/18365 proposes an edible film which should dissolve rapidly, but can also adhere well to the oral mucosa so as to dispense antimicrobial substances, and which reduces the number of undesirable microorganisms in the oral flora. The antimicrobial substances are essential oils which are mixed as lipophilic phase preferably with pullulan as matrix material in the aqueous phase.

US 2001/006677 discloses film-like, foaming dosage forms which are soluble or swellable in water and easily adhere to the oral mucosa.

WO 02/02085 describes rapidly disintegrating dosage forms for releasing active ingredients in the oral cavity or other bodily orifices, wherein the dosage form has a matrix which contains at least one water-soluble polymer as base substance and which is provided with cavities.

DE 10 2005 058 569 describes a foam wafer product for releasing active ingredients, such as especially nicotine, in the oral cavity which is based on a polyvinyl alcohol-polyethylene glycol graft copolymer.

The mode of action of the above-described wafers is based on the fact that that polymers used as matrix for the particular active ingredient dissolve upon contact with water or saliva, and the wafer thus disintegrates, wherein the active ingredients are released. The occurrence and the temporal progression of the active ingredient release is highly dependent on the thickness of the wafer. The thinner the wafer, the quicker the disintegration in an aqueous environment, since the solvent can penetrate more quickly inwardly into the dosage form. On the other hand, corresponding wafers must have a certain minimum thickness so as to be able to perform their intended function and so as to offer sufficient ease of handling. In addition, the thickness of such dosage forms is dependent on the type and amount of active ingredient that they are intended to contain and release. With increasing thickness, the disintegration or dissolution of the wafer is slowed accordingly.

On account of their flat, smooth form, thicker wafers especially, but also those with a relatively small thickness, with delayed disintegration tend to adhere and stick to the roof of the mouth or to other mucous membrane surfaces of the oral cavity. This is caused, inter alia, by the polymer layers that dissolve at the surface and that form a tacky and mushy film. This has caused wafer systems to be formulated nowadays generally with active ingredients of which only small amounts have to be used, since such greater thicknesses, which are caused by large active ingredient amounts, can be avoided. Due to the physicochemical properties necessary for such films (for example sufficient strength), there is thus an increasing reliance on conventional delivery systems, such as tablets, for active ingredients that have to be administered in larger amounts. A further problem of thicker wafers is the delayed release of the active ingredient or other constituents, such as especially taste-masking agents. If these are present in the form of insoluble or poorly soluble solids, these solids remain longer in the mouth due to the slower dissolution behaviour, and this may be perceived as unpleasant. In order to improve the sensation produced in the oral cavity by the wafer, a planar dosage form that rapidly disintegrates or rapidly dissolves in an aqueous environment and has spaces or cavities in a polymer matrix of the dosage form was proposed in the above-mentioned document WO 02/02085, wherein the content of the spaces/cavities differs in respect of its state of aggregation from that of the matrix.

Tests, however, have shown that sensitive individuals experience an unpleasant or annoying sensation in the oral cavity, also when a planar dosage form according to the teaching of WO 02/02085 is ingested. With regard to the dissolution behaviour in films, which have to have a high mass per unit area on account of the active ingredient amount necessary for an effective dose, there is thus a need for an improved dosage form, especially of a wafer, which does not lead to an unpleasant sensation in the mouth caused by the dissolution of the dosage form, especially of the wafer.

The present invention addresses this need.

### DETAILED DESCRIPTION OF THE INVENTION

In order to solve the above-described problem, the present invention according to claim 1 describes a planar dosage form that disintegrates or dissolves in an aqueous environment for releasing at least one active ingredient in a body orifice or body cavity, and that comprises a polymer matrix in the form of a solidified foam having cavities, and at least one pharmaceutical active ingredient, wherein the dosage form has a mass per unit area in the range of 50 to 350 g/m². The dosage form according to the invention consequently has spatial regions which are filled with a gas, such as especially air or nitrogen, and which provide the dosage form with an accelerated dissolution behaviour. The cavities may be situated only within the polymer matrix, but may also extend as far as the outer edge of the dosage form.

Due to the cavities according to the invention and the associated larger surface of the films, especially the ingress of water or saliva or other bodily fluids into the dosage form is facilitated, and therefore the dissolution of the dosage form and the active ingredient release are accelerated to such an extent that any active ingredient particles not easily soluble can rapidly disseminate in the mouth and throat and therefore are no longer perceptible. Such particles are thus prevented from remaining in situ, and a released active ingredient can be quickly swallowed. This results in an improved “mouthfeel” for the dosage form according to the invention, which ultimately leads to an improved acceptance among users or patients. In the event of a sublingual application of the active ingredient, the dosage form according to the invention additionally makes it possible for the active ingredient to be provided more quickly for transmucosal uptake.

With a rapidly resorbing active ingredient, the transmucosal resorption may be improved additionally by the rapid dissolution of the dosage form, for example in the case of sublingual application. On the other hand, the wall thickness of the mentioned cavities is low, since these for example represent solidified bubbles, and thus these cavities dissolve or disintegrate rapidly.

A further advantage of the dosage form according to the invention lies in the fact that, in spite of the relatively high mass per unit area, quicker drying can be realised by the formulation in foam form than in the case of a comparable non-foamed composition.

A range of 50 to 300 g/m², especially a range of 100 to 280 g/m², preferably a range of 130 to 250 g/m², more preferably a range of 150 to 220 g/m², and most preferably a range of 165 to 210 g/m² can be specified as a preferred mass per unit area for the dosage form according to the invention.

Water-soluble polymers or mixtures of such polymers are used as matrix polymers. In this regard, emulsifying synthetic or partially synthetic polymers or biopolymers of natural origin, which are film-forming and water-soluble, and/or which are suitable for forming foam are preferably used. Alternatively, polymers can be used which are not emulsifying per se when used in combination with surfactants. Suitable synthetic polymers are, for example, polyvinyl alcohol, polyacrylates and polyvinylpyrrolidone. Of these, polyvinyl alcohol is especially suitable. A very especially well-suited polyvinyl alcohol has a weight-average molecular weight in the range of 15,000 to 60,000 and especially in the range of 25,000 to 50,000. An example of a suitable commercial polyvinyl alcohol is Mowiol 4-88, which is sold for example by Sigma Aldrich.

Besides the above-mentioned homopolymers, copolymers may also be used as synthetic polymers. Suitable copolymers are, for example, polyvinyl alcohol-polyethylene glycol graft copolymers, such as those obtainable under the trade names Kollicoat® IR from BASF, or polyvinylpyrrolidone-polyvinyl acetate copolymers, such as those obtainable under the trade name Kollidon VA 64, from BASF.

Suitable partially synthetic polymers are cellulose derivatives, such as hydroxypropyl methylcellulose, carboxymethyl cellulose, hydroxypropyl cellulose, hydroxymethyl cellulose and methyl cellulose, as well as other substituted cellulose derivatives. A especially suitable cellulose derivative is hydroxypropyl methyl cellulose, especially hydroxypropyl methyl cellulose with a degree of methoxy substitution of approximately 28 to 30% and a degree of hydroxypropoxy substitution of approximately 7 to 12%. Such a hydroxypropyl methylcellulose is obtainable for example under the trade name Methocel E from Dow Chemical. Water-soluble polysaccharides which are of plant, microbial or synthetic origin, especially polysaccharides which are not cellulose derivatives, such as pullulan, xanthan, alginates, dextrans, agar-agar, pectins, and carrageenan, are also preferred. Furthermore, proteins, preferably gelatines or other gel-forming proteins, and protein hydrolysates, are also suitable. Suitable protein hydrolysates include, inter alia, caseinate, whey, and plant proteins, gelatines, and (chicken) egg white and mixtures thereof. Preferred proteins are caseinates, which originate from spray-dried milk products.

Matrix polymers that are especially preferred within the scope of the present invention are polyvinyl alcohol, a polyvinyl alcohol-polyethylene glycol graft copolymer, and hydroxypropyl methyl cellulose. Polyvinyl alcohol is most preferred. The polyvinyl alcohol is preferably the hydrolysis product of a polyvinyl acetate homopolymer. It may contain residual amounts of preferably no more than 20 mol % and especially no more than 15 mol % (based on the total molar amount of vinyl alcohol and vinyl acetate monomers) of non-hydrolysed polyvinyl acetate. The above-mentioned preferred matrix polymers have the advantage that an addition of further surface-active ingredients or surfactants is not necessary for production of a solidified foam.

The matrix polymer in the dosage form according to the invention constitutes not only the pharmaceutical active ingredient, but also a main constituent of the dosage form. A content of 20 to 65% by weight, especially 30 to 60% by weight, and most preferably 32 to 52% by weight, based on the dry weight, may be specified as suitable proportion for the matrix polymer.

As already mentioned above, the cavities in the dosage form according to the invention may be present in the polymer matrix isolated from one another, preferably in the form of solidified bubbles.

In accordance with another embodiment it is provided that the cavities are connected to one another, preferably by forming a cohesive channel system penetrating the matrix.

The aforementioned cavities are preferably filled with gas or a gas mixture, especially air or nitrogen. In addition, however, it may also be advantageous if the cavities contain other gases or gas mixtures which do not react with other constituents of the dosage form. Especially preferred gases are nitrogen, carbon dioxide, and helium, and also a mixture of these gases or of a plurality of these gases.

The aforesaid cavities preferably have a volume fraction of 5 to 98%, preferably 50 to 80%, based on the total volume of the dosage form. The intended effect of the accelerated dissolution of the dosage form is in this way influenced favourably.

A further important parameter that influences the properties of the dosage form according to the invention is the diameter of the cavities or bubbles. The bubbles or cavities are preferably produced with the aid of a foaming machine, with which the diameter of the bubbles can be set within a wide range, almost arbitrarily. The diameter of the bubbles or cavities may thus lie in a range of 0.01 to 60 μm. The diameter especially preferably lies in a range of 10 to 50 μm.

The surface of the dosage form may be flat, however it is also possible for the surface to be uneven or irregularly shaped, for example corrugated or relief-like. Such an irregular surface structure may be caused for example by the bubble-like cavities formed in the polymer matrix, and/or by a subsequent drying treatment,

The dosage forms according to the invention are preferably provided in a thin design, for example in the form of a wafer. The thickness of the dosage form is preferably between 100 μm and 5 mm, especially preferably between 0.5 and 3 mm.

With regard to the pharmaceutical active ingredient, the present invention is not subject to any relevant limitations, with the exception that oral resorption, for example transmucosal, sublingual, or gingival resorption, and/or gastrointestinal resorption of the active ingredient must be possible.

Suitable active ingredients are consequently, inter alia, agents for treating infection; virostatics; analgesics such as fentanyl, sufentanil, buprenorphine; anaesthetics; anorectics; active ingredients for the treatment of arthritis and asthma, such as terbutaline; anticonvulsants; antidepressants; antidiabetics; antihistamines; antidiarrhoeics; agents against migraines, itching, sickness and nausea; travel and sea sickness, such as scopolamine and ondansetron; Parkinson's drugs; antipsychotics; antipyretics, spasmolytics, anticholinergics, agents against ulcers, such as rantidines, sympathomimetics; calcium channel blockers such as nifedipine; betablockers; beta agonists such as dobutamine; antiarrhythmics; antihypertonics such as atenolol; ACE inhibitors such as enalapril; benzodiazepine agonists such as flumazenil; coronary, peripheral and cerebral vasodilators; stimulation for the central nervous system; hormones; hypnotics; immunosuppressants; muscle relaxants; N-methyl D aspartate (NMDA) receptor antagonists; parasympatholytics; parasympathomimetics; prostaglandins; proteins, peptides; psychostimulants; sedatives; tranquilisers; adrenalin.

Active ingredients that are especially preferred within the scope of the present invention are N-methyl-D-aspartate (NMDA) receptor antagonists, especially in the form of dextromethorphan or ketamine or a pharmaceutically active derivative thereof. The ketamine may be used as racemate, however it is preferred if the ketamine is incorporated as S-ketamine in the dosage form according to the invention. Suitable pharmaceutically effective derivatives of ketamine are, for example, nor-S-ketamine, S-dehydronorketamine, or (S,S)-6-hydroxynorketamine. The use of pharmaceutically acceptable salts of the mentioned active ingredients is also included by the present invention.

The active ingredient content per dosing unit is up to 100 mg, preferably up to 50 mg, especially preferably up to 30 mg, and most preferably up to 20 mg. On the other hand, the minimum active ingredient content per dosing unit should preferably be 5 mg, more preferably 10 mg, and most preferably 12 mg. Depending on the application, the active ingredient amount may also lie in the upper range of the above values, for example in the range of more than 50 to 100 mg or 30 to 50 mg.

The active ingredient amount, set in relation to the area of the dosage form, is expediently in the range of 1 to 15 mg/cm², and preferably 2.8 to 10 mg/cm².

The active ingredient content in the dosage form according to the invention may vary within relatively wide limits. A content range from 20 to 60% by weight, based on the dry weight of the dosage form, may be specified as suitable. In one embodiment the proportion of active ingredient in the dosage form lies rather in the lower range, for example if the active ingredient has a strong unpleasant taste, which has to be compensated for by a larger amount of taste-masking agents. In this case a range of 21 to 30% by weight, and especially 22 to 28% by weight may be specified as suitable active ingredient proportion. In another embodiment the proportion of active ingredient in the dosage form according to the invention lies rather in the upper range, wherein a content of 42 to 55% by weight and especially a content of 45 to 52% by weight may be specified as especially preferred.

Besides the pharmaceutical active ingredient, the dosage form according to the invention may also contain further additives, for example so as to influence the colour or taste sensation when the dosage form is ingested.

An additive that is especially suitable in this regard is a taste-masking agent which contributes to an improved taste sensation, for example when bitter-tasting active ingredients are ingested. A preferred taste-masking agent is preferably an ion exchange resin.

Ion exchange resins that are preferred for use in the dosage form according to the invention are insoluble in water and consist of a pharmacologically inert organic or inorganic matrix which contains covalently bonded functional groups that are ionic or can be ionised under the suitable conditions of the pH value. The organic matrix may be synthetic (for example polymers or copolymers of acrylic acid, methacrylic acid, sulfonated styrene, sulfonated divinylbenzene) or partially synthetic (for example modified cellulose and dextrans). The matrix may also be inorganic, for example silica gel, modified by addition of ionic groups. The covalently bonded ion groups may be heavily acidic (for example sulfonic acid). weakly acidic (for example carboxylic acid), heavily alkaline (for example quaternary ammonium), weakly alkaline (for example primary amine) or a combination of acidic and alkaline groups. Those types of ion exchangers that are suitable for use in ion exchange chromatography and for applications such as deionisation of water are generally suitable for use in the dosage forms according to the invention.

The ion exchange resin is preferably a resin based on crosslinked polystyrene. The polystyrene is crosslinked with a crosslinking agent that is selected from difunctional compounds which are able to crosslink polystyrenes. The crosslinking agent is preferably a divinyl or polyvinyl compound. The crosslinking agent is most preferably divinylbenzene.

The polystyrene is generally crosslinked expediently to an extent of approximately 3 to approximately 20%, preferably approximately 4 to approximately 16%, more preferably approximately 6 to approximately 10%, and most preferably approximately 8% by weight, based on the total polystyrene. The polystyrene is crosslinked with the crosslinking agent by known means.

Within the scope of the present invention, ion exchange resins that are especially suitable as taste-masking agents have exchange capacities below approximately 6 milliequivalents per gram (meq/g) and preferably below approximately 5.5 meq/g.

The size of the ion exchange particles should preferably fall in the range of approximately 20 to approximately 200 micrometres. Particle sizes considerably below the lower limit are difficult to handle in all steps of the processing. Particle sizes substantially above the upper limit, for example commercially obtainable ion exchange resins of spherical form and diameters up to approximately 1000 micrometres, are gritty in liquid dosage forms and have a tendency to fracture when exposed to dry hydration cycles.

Representative resins that are usable in this invention comprise AMBERLITE IRP-69 (obtainable from Dow Chemical) and Dow XYS-40010.00 (obtainable from the Dow Chemical Company). Both are sulfonated polymers formed from polystyrene, crosslinked with 8% divinylbenzene, with an ion exchange capacity of approximately 4.5 to 5.5 meq/g dry resin (H+form). Their main difference lies in their physical form. AMBERLITE IRP-69 comprises irregularly shaped particles with a size range of 47 to 149 micrometres, produced by milling the superordinate, large-area spheres of AMBERLITE IRP-120. The Dow XYS-40010.00 product comprises spherical particles with a size range of 45 to 150 micrometres. A further usable exchange resin, Dow XYS-40013.00, is a polymer that consists of polystyrene crosslinked with 8% divinylbenzene and functionalised with a quaternary ammonium group. Its exchange capacity normally lies in the range of approximately 3 to 4 meq/g dry resin. A further suitable resin is AMBERLITE IRP-64.

In less preferred embodiments the taste-masking agent, however, does not need to be an ion exchange resin. In these embodiments the taste-masking agent may be magnesium trisilicate or a polymer such as EUDRAGIT E (Evonik) and/or cellulose, such as ethyl cellulose or the like.

The content of taste masking agent that is incorporated in the dosage form according to the invention is based on whether the pharmaceutical active ingredient has an unpleasant, for example bitter taste. The content of taste-masking agent generally moves within a range of 0.3 to 45% by weight, and especially in a range of 0.5 to 27% by weight, in each case based on the dry weight of the dosage form.

In order to modify the taste sensation, the dosage form according to the invention may contain a sweetener in addition to a taste-masking agent, or alternatively. Suitable synthetic sweeteners are, for example, sucralose, aspartame, cyclamate, saccharine, neohesperidin, thaumatin, stevia and acelsulfame, and salts thereof.

The dosage form according to the invention may also contain one or more flavourings, essential oils, or menthol.

When producing the dosage form according to the invention, one or more acids may additionally be admixed, so as to give the foam a pleasant acidic flavour. Examples of such acids include, inter alia, citric acid, lactic acid, acetic acid, benzoic acid, propionic acid, oxalic acid, malonic acid, succinic acid, malic acid, and tartaric acid. The addition of acid(s) may additionally be necessary or desirable in order to reduce the pH value of the foam. This is desirable especially if the active ingredient contained in the dosage form is relatively insoluble under alkaline conditions, or in the case of active ingredients that are not stable in alkaline conditions.

Especially foams, damping agents or humectants and/or plasticisers may also be added to the dosage form according to the invention in order to improve the aesthetic properties of the dried foam and reduce the fragility or brittleness of the dried foam. Examples of such agents are, inter alia, glycerol, propylene glycol and polyglycerol ester. The content of plasticiser varies expediently in a range from 2 to 10% by weight, especially 3 to 8% by weight, and most preferably 4 to 6% by weight, based on the dry weight of the dosage form.

In one embodiment, surface-active ingredients or surfactants may be added to the matrix polymer or the polymer matrix for foam formation or to the obtained foam before or after the drying, in order to improve the stability of the foam before or after the drying. Inter alia, substituted sorbitan derivatives, especially those from the “Tween” range (ICI) or “Span” range (TCI), can be considered as examples of suitable surface-active ingredients. Surface-active ingredients may also be present in the form of foam-stabilising polymers (for example cellulose-based polymers or polyacrylate-based polymers, which were not listed above as matrix polymers), or in the form of casein or gelatine.

The proportion of the surfactant in the dosage form according to the invention is dependent primarily on whether the matrix polymer requires a surfactant for the stabilisation of a foam, which is not the case for example with emulsifying matrix polymers, such as polyvinyl alcohol. A range of 0 to 15% by weight, based on the dry weight of the dosage form, can be specified as suitable surfactant proportion for the dosage forms according to the invention.

In one embodiment the matrix polymer does not require any surface-active ingredient or surfactant for the stabilisation of a foam. In this case the proportion of surface-active ingredients and/or surfactant in the dosage form according to the invention is preferably less than 0.5% by weight and especially preferably less than 0.1% by weight, based on the dry weight of the dosage form. It should be noted here that the substances listed above as matrix polymers, in the context of this invention, are not considered to be surface-active ingredients, although some of these substances have surface-active properties. This is even advantageous, since in this case the above-listed surface-active ingredients or surfactants do not have to be incorporated in the foam composition.

In order to give the dosage form a desired colour, a dye or colorant can be incorporated into the dosage form. Suitable dyes are, for example, azo dyes such as Allurarot AC, which is also obtainable under the trade name FD&C Red 40.

Inter alia, substances from the following group are potential further additives: carboxymethyl cellulose, gum arabic, methyl cellulose, pectins, modified and non-modified starches, gelatine, animal and/or plant proteins, chicken egg white, alginate, Brij (an emulsifier), ethyl citrate, octyl gallate, 1,2-propylene glycate, magnesium stearate, stearic acid, microcrystalline cellulose, Aerosil, lecithin, Tween, propyl gallate, amylogam.

In addition, a sugar (or a mixture of sugars) or another carbohydrate material may be dissolved in the foam. The sugar or the carbohydrate increases the mass that the foam has after drying. In addition, the drying and the crystallisation of the sugar or another carbohydrate give the dried foam an additional strength and stability. The sugar or other carbohydrates may bring about a sweet taste of the dried foam or may otherwise improve the organoleptic properties of the foam. Examples of sugars usable for this purpose are, inter alia, maltose, lactose, sucrose, dextrose (glucose) and trehalose, as well as sugar alcohols, such as mannitol, sorbitol, xylitol, maltitol and the like. Examples of other carbohydrates are maltodextrin, glucose syrup (from corn), soluble starches, and the like.

The content of additives, insofar as no specific content values have been specified above, should lie in the range of 0.01 to 10% by weight, and especially 0.1 to 8% by weight, based on the dry weight of the dosage form.

In addition to the aforementioned constituents, the dosage form according to the invention may contain moisture (water). A proportion in the range of 2 to 15% by weight, and especially 5 to 12% by weight, is specified as suitable moisture content.

The invention is intended primarily for use as an oral dosage form which release active ingredients in the region of the oral cavity. However, it can also be used as a dosage form that is introduced into other body orifices or body cavities and releases its active ingredients there. In this respect, rectal, vaginal or intranasal dosage forms are possible, for example.

The active ingredient released from the dosage form is either resorbed at the application site, for example via the oral mucosa, or is transported on further and resorbed at another location (for example in the gastrointestinal tract once the active ingredient released in the oral cavity has been swallowed). The time for which the dosage form according to the invention remains at the application site (for example oral cavity) or the disintegration time lies preferably in the range of 1 s to 5 min, more preferably in the range of 2 s to 1 min, even more preferably in the range of 3 to 10 s, and most preferably in the range of 3 to 5 s.

If the dosage form contains ketamine or S-ketamine as active ingredient, this may be used expediently for the treatment of complaints for which ketamine or S-ketamine contribute to relief. A further aspect of the present invention therefore relates to a dosage form according to the above specifications with a content of ketamine or S-ketamine or pharmaceutically acceptable salts thereof for the therapeutic or prophylactic treatment of pain, preferably of chronic pain, and more preferably pain selected from the group comprising chronic breakthrough pain, complex regional pain syndrome, resistant tumour pain, neuropathic pain, post-traumatic syndrome pain, ischaemic limb pain and acute pain. Alternatively, the present invention relates to a dosage form according to the above specifications with a content of ketamine or S-ketamine or pharmaceutically acceptable salts thereof for the therapeutic or prophylactic treatment of depression. In these dosage forms a sublingual application is especially preferred, since this ensures a rapid availability of ketamine for transmucosal uptake.

If the dosage form contains dextromethorphan as active ingredient, it may be used expediently for the treatment of complaints for which dextromethorphan contributes to relief. A further aspect of the present invention therefore relates to a dosage form according to the above specifications with a content of dextromethorphan or a pharmaceutically acceptable salt thereof for the therapeutic or prophylactic treatment of coughs, emotional dysregulation disorders, or amyotrophic lateral sclerosis.

If the dosage form contains adrenalin as active ingredient, it may be used expediently for the treatment of complaints for which adrenalin contributes to relief. A further aspect of the present invention therefore relates to a dosage form according to the above specifications with a content of adrenalin or a pharmaceutically acceptable salt thereof for the therapeutic or prophylactic treatment of anaphylactic shock.

The dosage forms according to the invention can be produced expediently with the aid of the methods described hereinafter.

Firstly, a solution or dispersion is produced which contains at least one water-soluble film-forming polymer and at least one active ingredient. This solution or dispersion, which may also be a concentrated solution or viscous mass, is then foamed by introducing gas or a gas mixture (for example air). This may be achieved by means of a dispersing mechanism or a foaming machine, but also by other methods, for example by means of ultrasound. Especially, inert gases, such as nitrogen, carbon dioxide or helium, or mixtures thereof, are also suitable as gases.

In order to stabilise the foams or air-bubble-containing (or gas-bubble-containing) masses thus produced, a foam-stabilising agent may be added before or during the foam production. Agents suitable for this purpose, for example surfactants, are known to a person skilled in the art. Lastly, the air-bubble-containing mass or the foam is spread in the form of a film or layer on a suitable substrate and is then dried.

The “drying” shall be understood in the sense that solvent, especially water, is removed from the dosage form. To this end, it is not necessary for all solvent, such as water, to be removed from the dosage form, but rather it is sufficient if the majority of solvent is removed from the dosage form, such that the foam solidifies. The dosage form, after the drying, may thus have a residual water content as specified above for the dosage form according to the invention.

As a result of the solvent removal, the foam solidifies during the drying, wherein the formed cavities maintain a permanent structure. Wafers with desired surface dimensions or geometric shapes are obtained by pouring the foamed coating mass into appropriate moulds before the drying, or by punching out the individual wafers from a larger two-dimensional piece.

The active-ingredient-containing dosage forms thus obtained have the properties and preferences according to the invention.

The shape, number and size of the produced cavities can be influenced by means of different method parameters, for example by the type and concentration of the polymers, by the viscosity of the polymer mass, by control of the foaming process, by selection of the foam-stabilising agents, etc.

Alternatively to the above-described method, it is possible to produce the dosage forms according to the invention via a method in which the cavities within the polymer matrix are formed by introducing a hydrophobic solvent not miscible with the solvent used for the production of the described solution or dispersion.

An emulsion is produced hereby, which contains the hydrophobic solvent in the form of finely dispersed droplets.

Due to the removal of the solvent during the subsequent drying, droplet-shaped or bubble-shaped cavities remain in the polymer matrix. In the case of a two-phase system, the solvent must firstly be removed from the inner phase.

In a modification of the above-described method, the described cavities may also be produced in such a way that auxiliaries are added to the polymer-containing and active-ingredient-containing solution or dispersion and form a gas or gases, whereby the mass is foamed. This foaming by gas development may occur either during the production of the polymer mass or during the coating of this mass on the substrate, or only during the subsequent drying process. Substances or substance mixtures suitable for gas formation are known to a person skilled in the art. The foaming may also be brought about by expansion of a previously dissolved gas. Especially an inert gas, such as nitrogen, carbon dioxide or helium, or a mixture thereof may be used as gas.

When producing the dosage forms according to the invention, it is also possible, alternatively, to start from a melt of the matrix polymer or polymer mixture. The processing is performed in principle similarly to that in the case of hot-melt coating masses known in the prior art.

A gas or a gas mixture is introduced into the described polymer melt by one of the above-described methods, so as to make the melt foam. The melt is then spread or extruded onto a suitable substrate, or is poured into a mould, and is then left to cool or solidify. It is not possible to process the melt if the active ingredient provided is unstable or volatile at the melting point of the polymer melt. If necessary, auxiliaries for reducing the melting point may be added to the polymer melt. In principle, hot-melt coating masses known from the prior art may also be used, provided they satisfy the conditions stated in claim 1.

In accordance with a further modification of the above-described production methods, the polymer matrix is produced firstly in the form of a block. The desired dosage form is separated from this block subsequently, i.e. after drying or solidification, by cutting.

The dosage form according to the invention is suitable advantageously for the administration of medicaments in the oral cavity or for rectal, vaginal or intranasal administration. They may be used in human medicine and also in veterinary medicine.

### PRODUCTION EXAMPLE

### Example 1

A foamed wafer with dextromethorphan was produced with the following composition:

| | |
|---|---|
| Polyvinyl alcohol (Mowiol 4-88) | 38.00% by weight |
| Red dye | 0.20% by weight |
| Sodium saccharin (sweetener) | 1.0% by weight |
| Sucralose (sweetener) | 2.0% by weight |
| Glycerol (plasticiser) | 4.5% by weight |
| Flavourings | 6.0% by weight |
| Active ingredient | 19.7% by weight |
| Taste-masking agent | 28.6% by weight |

For production of a solidified foam, a pre-solution of the polyvinyl acetate in water was firstly produced, in which the active ingredient was dispersed. The dispersion was then foamed with inclusion of air and was applied to a polyethylene carrier film with the aid of a doctor blade/coating box. The foam was then dried in a drying cabinet for 15 min at approximately 70° C. so as to obtain a solidified foam. The foam thus produced had a mass per unit area of 200 g/m².

### Example 2

The composition specified in Table 1 below was processed in accordance with the specifications described in Example 1 with use of Ketamine HCl as active ingredient to form a solidified foam. In parallel, the composition was processed without foaming to form a comparison film.

TABLE 1

| | 1 |
|---|---|
| Active ingredient [g] | 50 |
| Polyvinyl alcohol (Mowiol 4-88) [g] | 38.3 |
| Red dye [g] | 0.2 |
| Sodium saccharin [g] | 1.0 |
| Sucralose [g] | 2.0 |
| Glycerol [g] | 4.5 |
| Flavourings [g] | 3.5 |
| Taste-masking agents [g] | 0.5 |
| Mass per unit area [g/m²] | 200 |

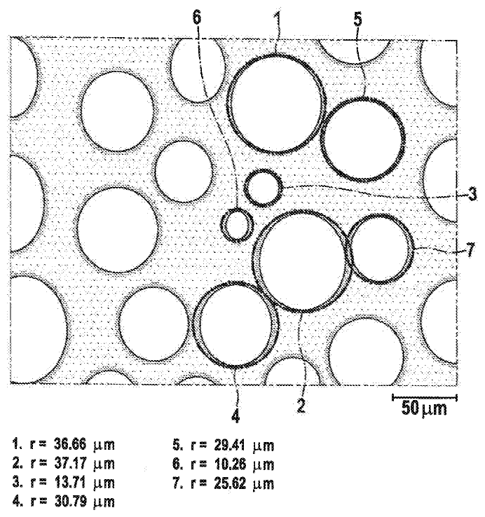

The solidified foam was examined under a microscope. A recorded microscope image is reproduced in FIG. 1 and shows that the films have cavities with diameters ranging from approximately 10 to 37 μm 50 μm. On the whole, cavity sizes in the range of 10 to 50 μm could be detected in the film.

The disintegration of the produced foams and comparison films was determined. Disintegration times of approximately 13 seconds (mean value from 6 measurements) were determined for the foams. The comparison films had much longer disintegration times of approximately 38 seconds.

1. A planar dosage form that disintegrates or dissolves in an aqueous environment for releasing at least one active ingredient in a body orifice or body cavity comprising
a polymer matrix in the form of a solidified foam having cavities and at least one pharmaceutical active ingredient,
wherein the dosage form has a mass per unit area in the range of 50 to 350 g/m².

2. The dosage form according to claim 1, wherein it the dosage form has a mass per unit area in the range of 50 to 300 g/m².

3. The dosage form according to claim 1, wherein the polymer matrix is based on a polymer selected from the group comprising polyvinyl alcohol, a polyvinyl alcohol-polyethylene glycol graft copolymer, and hydroxypropyl methylcellulose.

4. The dosage form according to claim 1, wherein the cavities are isolated from one another and are preferably present in the form of bubbles.

5. The dosage form according to claim 1, wherein the cavities are connected to one another and preferably form a channel system penetrating the polymer matrix.

6. The dosage form according to claim 1, wherein the cavities are filled with air or a gas, or a mixture of these gases.

7. The dosage form according to claim 1, wherein said cavities have a volume fraction of 5 to 98%, based on the total volume of the dosage form.

8. The dosage form according to claim 1, wherein the dosage form is formed as a wafer.

9. The dosage form according to claim 1, that wherein the pharmaceutical active ingredient contains ketamine, dextromethorphan, or a pharmaceutically acceptable salt thereof.

10. The dosage form according to claim 1, wherein the pharmaceutical active ingredient accounts for a proportion in the range of 38 to 60% by weight, based on the total weight of the dosage form.

11. The dosage form according to claim 1, wherein the dosage form contains a taste-masking constituent.

12. A method for producing a dosage form according to claim 1, wherein said method comprises
producing a solution or dispersion which contains at least one matrix polymer and at least one pharmaceutical active ingredient;
foaming of the solution or dispersion by introduction of a gas or gas mixture, or by chemical gas generation, or by expansion of a dissolved gas, optionally after prior addition of a foam-stabilising agent;
spreading of the foamed solution or dispersion onto a coating substrate; and
solidification of the spread solution or dispersion by drying and removal of the solvent.

13. A method for producing a dosage form according to claim 14, wherein said method comprises
a) producing a solution or dispersion which contains at least one matrix polymer and at least one pharmaceutical active ingredient;
b) adding an auxiliary or a combination of auxiliaries which are capable of gas formation;
c) spreading the solution or dispersion onto a coating substrate; and
d) solidifying the spread solution or dispersion by drying and removal of the solvent.

14. A method for producing a dosage form according to claim 1, wherein said method comprises
a) producing a polymer-containing hot melt which contains at least one matrix polymer and at least one pharmaceutical active ingredient;
b) foaming the melt by introducing a gas or gas mixture, or by chemical gas generation, or by expansion of a dissolved gas, optionally after prior addition of a foam-stabilising agent;
c) spreading of the melt onto a coating substrate; and
d) solidifying the film by cooling.

15. The method according to claim 13, wherein steps c) and d) are replaced or modified by the following steps e) and f):
e) producing the polymer matrix in the form of a block starting from the solution or dispersion or from the melt;
f) cutting the solidified block into planar forms.

16. A method of administering pharmaceutical active ingredients comprising administering a dosage form according to claim 1 in the oral cavity, rectum, vagina or intranasally.

17. (canceled)

18. The dosage form according to claim 9 wherein the pharmaceutical active ingredient contains a content of ketamine or a pharmaceutically acceptable salt thereof for the therapeutic or prophylactic treatment of pain or depression.

19. The dosage form according to claim 9 wherein the pharmaceutical active ingredient contains a content of dextromethorphan or a pharmaceutically acceptable salt thereof for the therapeutic or prophylactic treatment of coughs, emotional dysregulation disorders, or amyotrophic lateral sclerosis.

20. The dosage form according to claim 1, wherein the dosage has a mass per unit area in the range of 130 to 250 g/m².

21. The dosage form according to claim 1, wherein the dosage has a mass per unit area in the range of 150 to 220 g/m².

22. The dosage form according to claim 1, wherein said dosage has a mass per unit area in the range of 165 to 210 g/m².

23. The dosage form according to claim 5, wherein the cavities form a channel system penetrating the polymer matrix.

24. The dosage form according to claim 6, wherein the cavities are filled with an inert gas.

25. The dosage form according to claim 6, wherein the cavities are filled with one or more of nitrogen, carbon dioxide or helium.

26. The dosage form according to claim 7, wherein said cavities have a volume fraction of 50 to 80%, based on the total volume of the dosage form.

27. The dosage form according to claim 8, wherein the dosage form has a thickness of between 100 μm and 5 mm.

28. The dosage form according to claim 8, wherein the dosage form has a thickness of between 0.5 and 3 mm.

29. The dosage form according to claim 9, wherein the ketamine is S-ketamine or a pharmaceutically acceptable salt thereof.

30. The dosage form according to claim 10, wherein the pharmaceutical active ingredient accounts for a proportion in the range of 42 to 55% by weight, based on the total weight of the dosage form.

31. The dosage form according to claim 10, wherein the pharmaceutical active ingredient accounts for a proportion in the range of 45 to 52% by weight, based on the total weight of the dosage form.

32. The dosage form according to claim 11, wherein the taste-masking constituent is an ion exchange resin.

33. The dosage form according to claim 18, wherein the pain is chronic pain.
